(19) United States
(12) Patent Application Publication
(10) Pub. No.: US 2005/0150764 A1
(43) Pub. Date: Jul. 14, 2005

(54) Tetrahydropyridazine derivatives and their use as pesticides

(76) Inventors: Fritz Maurer, Lauffen (DE); Christoph Erdelen, Leichlingen (DE); Angelika Lubos-Erdenlen, Leichlingen (DE); Udo Reckmann, Koln (DE); Andreas Turberg, Haan (DE)

Correspondence Address: BAYER CROPSCIENCE LP;Patent Department, 100 BAYER ROAD, PITTSBURGH, PA, 15205-9741, US

(21) Appl. No.: 10/484,535

(22) PCT Filed: Jul. 12, 2002
(86) PCT No.: PCT/EP02/07780

(30) Foreign Application Priority Data
Jul. 25, 2001 (DE) 101 36 066.5

(51) Int. Cl. C07K 1/26

(52) U.S. Cl. 204/450

(57) ABSTRACT

The present invention relates to novel tetrahydropyridazine derivatives of the formula (I) 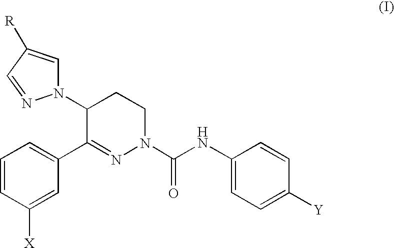 in which R, X and Y are as defined in the disclosure, to a process for preparing them and to their use as pesticides.

The present invention relates to novel tetrahydropyridazine derivatives, to a process for preparing them and to their use as pesticides.

It is known that certain tetrahydropyridazinecarboxamides exhibit good activity against animal pests (cf. e.g. DE-A 43 03 658 or WO 91/17-983).

The extent and/or duration of the action of these known compounds is, however, not entirely satisfactory in all areas of application, especially for certain organisms or at low concentrations

This invention provides novel tetrahydropyridazine derivatives of the formula (I) 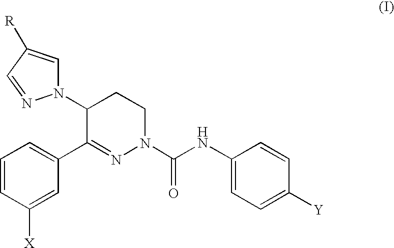
in which
R represents hydrogen, halogen, alkyl, alkoxy, alkylthio, haloalkyl, haloalkoxy, haloalkylthio, hydroxyl, nitro, cyano, alkoxycarbonyl, aminocarbonyl, alkylaminocarbonyl or dialkylaminocarbonyl,
X represents halogen, haloalkyl, haloalkoxy, alkylthio, alkylsulphinyl, alkylsulphonyl, haloalkylthio, haloalkylsulphinyl, haloalkylsulphonyl or cyano and
Y represents halogen, haloalkyl, haloalkoxy, haloalkylthio, halosulphinyl, haloalkylsulphonyl or cyano.

It has also been found that the tetrahydropyridazine derivatives of the formula (I) are obtained if tetrahydropyridazines of the formula (II) 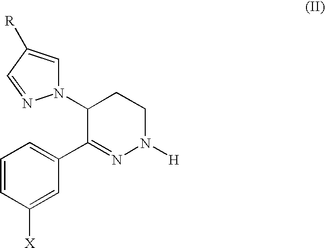
in which
R and X are as defined above
are reacted with isocyanates of the formula (III) 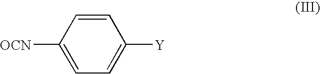
   in which
Y is as defined above,
optionally in the presence of a diluent.

Finally it has been found that the novel tetrahydropyridazine derivatives of the formula (I) possess strongly pronounced biological properties and are suitable in particular for controlling animal pests, especially insects, arachnids and nematodes, which occur in agriculture, forestry, the protection of stored products and materials, and in the hygiene sector.

A general definition of the tetrahydropyridazine derivatives of the invention is given by the formula (I).

Preferred substituents and ranges for the radicals set out in the formulae mentioned above and below are elucidated in the following text:
R preferably represents hydrogen, halogen, C₁-C₄-alkyl, C₁-C₄-alkoxy, C₁-C₄-alkylthio; represents C₁-C₄-haloalkyl, C₁-C₄-haloalkoxy and C₁-C₄-haloalkylthio having in each case from 1 to 5 identical or different halogen atoms from the series consisting of fluorine, chlorine and bromine; represents hydroxyl, nitro, cyano; represents C₁-C₄-alkoxy-carbonyl, aminocarbonyl, C₁-C₄-alkylamino-carbonyl or di-C₁-C₄-alkylamino-carbonyl.
X preferably represents halogen, C₁-C₄-alkylthio, C₁-C₄-alkylsulphinyl, C₁-C₄-alkylsulphonyl; represents C₁-C₄-haloalkyl, C₁-C₄-haloalkoxy, C₁-C₄-haloalkylthio, C₁-C₄-haloalkylsulphinyl and C₁-C₄-haloalkylsulphonyl having in each case from 1 to 5 identical or different halogen atoms from the series consisting of fluorine, chlorine and bromine; or represents cyano.
Y preferably represents halogen; represents C₁-C₄-haloalkyl, C₁-C₄-haloalkoxy, C₁-C₄-haloalkylthio, C₁-C₄-haloalkylsulphinyl and C₁-C₄-haloalkylsulphonyl having in each case from 1 to 5 identical or different halogen atoms from the series consisting of fluorine, chlorine and bromine; or represents cyano.
R with particular preference represents hydrogen, fluorine, chlorine, bromine, methyl, methoxy, methylthio, trifluoromethyl, trifluoromethoxy, trifluoromethylthio, cyano, methoxycarbonyl, ethoxycarbonyl, aminocarbonyl, methylaminocarbonyl, ethylaminocarbonyl, dimethylaminocarbonyl or methylethylaminocarbonyl.
X with particular preference represents fluorine, chlorine, bromine, trifluoromethyl, trifluoromethoxy, methylthio, methylsulphonyl, trifluoromethylthio, trifluoromethylsulphonyl or cyano.
Y with particular preference represents fluorine, chlorine, bromine, trifluoromethyl, trifluoromethoxy, trifluoromethylthio, trifluoromethylsulphonyl, difluoromethyl, difluoromethoxy or cyano.

The radical definitions and elucidations set out above or in ranges of preference apply to the end products and to the precursors and intermediates correspondingly. These definitions of radicals may be combined as desired with one another, hence including combinations between the respective ranges of preference.

Preference in accordance with the invention is given to the compounds of the formula (I) in which there is a combination of the definitions set out above as being preferred (preferably).

Particular preference in accordance with the invention is given to the compounds of the formula (I) in which there is a combination of the definitions set out above as being particularly preferred (with particular preference).

In the definitions of radicals set out above and below, hydrocarbon radicals, such as alkyl, both alone and in conjunction with heteroatoms, such as in alkoxy, are, where possible, in each case straight-chain or branched.

Using, for example, 4-(4-chloropyrazol-1-yl)-3-(3-fluorophenyl)-1,4,5,6-tetrahydropyridazine and 4-trifluoromethylphenyl isocyanate as starting materials, the course of the reaction in the process of the invention may be represented by the following formula scheme:

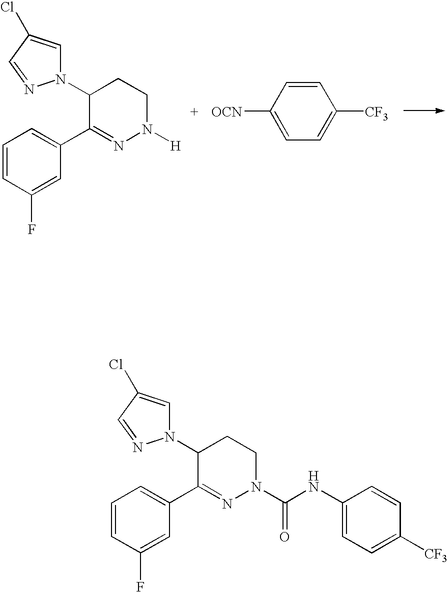

A general definition of the tetrahydropyridazines for use as starting materials in the process of the invention is given by the formula (II). The tetrahydropyridazines of the formula (II) were previously unknown and are likewise subject matter of this specification. They may be obtained conventionally by reacting ω-chloro ketones of the formula (IV) 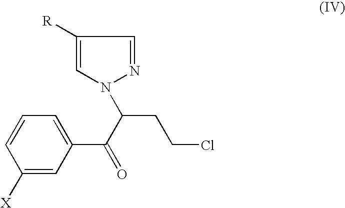
in which
R and X are as defined above
with hydrazine hydrate (NH₂—NH₂xH₂O), optionally in the presence of a diluent, such as ethanol, for example, at temperatures between 0° C. and 50° C. (cf. also the Preparation Examples).

The ω-chloro ketones of the formula (IV) are likewise novel and subject matter of this specification. They may be obtained conventionally by reacting dihalo ketones of the formula (V) 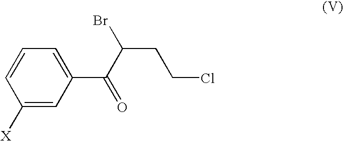
in which
X is as defined above
with known pyrazoles of the formula (VI) 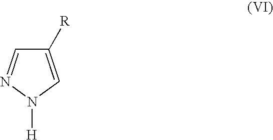
   in which
R is as defined above;
preferably in the form of the hydrohalides, such as hydrochlorides, for example optionally in the presence of an inert diluent, preferably nitrites, such as acetonitrile; ketones, such as acetone; or amides, such as dimethylformamide, and optionally in the presence of bases, such as alkaline earth metal carbamates, tert-alkoxides, alkali metal hydrides or tert-amines at temperatures from −20° C. to 40° C. (cf. also the Preparation Examples).

The dihalo ketones of the formula (V) are known (cf. e.g. EP-A 657 421) and/or obtainable by known methods by, for example, brominating the corresponding monohalo ketones of the formula (VI) 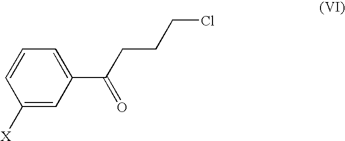
in which
X is as defined above
conventionally (cf. also the Preparation Examples).

The monohalo ketones of the formula (VI) are known (cf. e.g. EP-A 657 421 or U.S. Pat. No. 3,859,290) and/or obtainable by known methods by reacting furanone derivatives of the formula (VII) 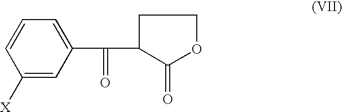
in which
X is as defined above
conventionally with concentrated hydrochloric acid at temperatures between 30° C. and 60° C. (cf. also the Preparation Examples).

Some of the furanone derivatives of the formula (VII) are known (cf. e.g. JP-A 55127382 [CA 94, 174852]). Hitherto unknown and likewise subject matter of this specification are furanone derivatives of the formula (VIIa) 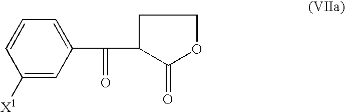
in which
X¹ represents fluorine, bromine; C₁-C₄-alkylthio, C₁-C₄-alkylsulphinyl, C₁-C₄-alkylsulphonyl; represents C₁-C₄-haloalkyl, C₁-C₄-haloalkoxy, C₁-C₄-haloalkylthio, C₁-C₄-haloalkylsulphinyl and C₁-C₄-haloalkylsulphonyl having in each case from 1 to 5 identical or different halogen atoms from the series consisting of fluorine, chlorine and bromine, or represents cyano.

The novel furanone derivatives of the formula (VIIa) may be obtained conventionally by reacting known methyl benzoates of the formula (VIII) 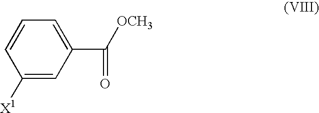
in which
X¹ is as defined above
with γ-butyrolactone of the formula (IX) 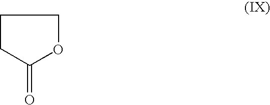
   conventionally in the presence of a diluent, such as tetrahydrofuran, for example, and in the presence of alkoxides, such as potassium tert-butoxide, for example, at temperatures between 0° C. and 80° C. (cf. also the Preparation Examples).

The isocyanates of the formula (III) also for use as starting materials in carrying out the process of the invention are common compounds of organic chemistry.

The process of the invention is preferably carried out using diluents. Suitable diluents include virtually all inert organic solvents. They include preferably aliphatic and aromatic, optionally halogenated hydrocarbons such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers such as diethyl and dibutyl ether, glycol dimethyl ether and diglycol dimethyl ether, tetrahydrofuran and dioxane, ketones such as acetone, methyl ethyl ketone, methyl isopropyl ketone or methyl isobutyl ketone, esters such as methyl or ethyl acetate, nitriles such as acetonitrile or propionitrile, for example, amides such as dimethylformamide, dimethylacetamide and N-methylpyrrolidone, for example, and also dimethyl sulphoxide, tetramethylene sulphone or hexamethylphosphoramide.

For the process of the invention, the reaction temperatures may be varied within a relatively wide range. It is normal to operate at temperatures between 0° C. and 100° C., preferably at temperatures between 10° C. and 80° C.

The process of the invention is generally carried out under atmospheric pressure. It is, however, also possible to operate under increased or reduced pressure.

For carrying out the process of the invention, the starting materials required in each case are generally used in approximately equimolar amounts. It is, however, also possible to use one of the two components used in each case in a relatively large excess. Workup in the processes of the invention takes place in each case in accordance with customary methods (cf. the Preparation Examples).

The active compounds are suitable for controlling animal pests, especially insects, arachnids and nematodes, which are encountered in agriculture, in forestry, in the protection of stored products and materials, and in the hygiene sector, and have good plant tolerance and favourable toxicity to warm-blooded animals. They may preferably be employed as plant protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp;, Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

The substances of the invention may be employed with particular success to control plant-damaging insects, such as against caterpillars of the cotton budworm (Heliothis virescens), the larvae of the mustard beetle (Phaedon cochleariae), the caterpillars of the cabbage moth (Plutella xylostella) and also caterpillars of the army worm (Spodoptera exigua and Spodoptera frugiperda).

Furthermore, the substances of the invention exhibit a resistance-inducing effect, particularly with regard to Erysiphe graminis.

If appropriate, the compounds according to the invention can, at certain concentrations or application rates, also be used as herbicides or microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

The treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds according to the invention can be converted into the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds according to the invention with extenders, that is to say liquid solvents and/or solid carriers, optionally with the use of surfactants, that is to say emulsifiers and/or dispersants and/or foam-formers.

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

Suitable solid carriers are:
for example ammonium salts and ground natural minerals such as kaolins, clays, talc, chalk, quartz, attapulgite, montmorillonite or diatomaceous earth, and ground synthetic minerals, such as highly disperse silica, alumina and silicates; suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95%; by weight of active compound, preferably between 0.5 and 90%.

The active compounds according to the invention can also be used as such or in their formulations as a mixture with known fungicides, bactericides, acaricides, nematicides or insecticides, in order to widen the activity spectrum or to prevent the development of resistance.

Particularly suitable co-components are, for example, the following compounds:

Fungicides:
aldimorph, ampropylfos, ampropylfos-potassium, andoprim, anilazine, azaconazole, azoxystrobin,
benalaxyl, benodanil, benomyl, benzamacril, benzamacryl-isobutyl, bialaphos, binapacryl, biphenyl, bitertanol, blasticidin-S, bromuconazole, bupirimate, buthiobate,
calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,
debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,
edifenphos, epoxiconazole, etaconazole, ethirimol, etridiazole,
famoxadon, fenapanil, fenarinmol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox,
guazatine,
hexachlorobenzene, hexaconazole, hymexazole,
imazalil, imibenconazole, iminoctadine, iminoctadine albesilate, iminoctadine triacetate, iodocarb, ipconazole, iprobenfos (IBP), iprodione, irumamycin, isoprothiolane, isovaledione,
kasugamycin, kresoxim-methyl, copper preparations, such as: copper hydroxide, copper naphthenate, copper oxychloride, copper sulphate, copper oxide, oxine-copper and Bordeaux mixture,
mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,
nickel dimethyldithiocarbamate, nitrothal-isopropyl, nuarimol,
ofurace, oxadixyl, oxamocarb, oxolinic acid, oxycarboxim, oxyfenthiin,
paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrinmethanil, pyroquilon, pyroxyfur,
quinconazole, quintozene (PCNB),
sulphur and sulphur preparations,
tebuconazole, tecloftalam, tecnazene, tetcyclasis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole,
uniconazole,
validamycin A, vinclozolin, viniconazole,
zarilamide, zineb, ziram and also
Dagger G,
OK-8705,
OK-8801,
α-(1,1-dimethylethyl)-β-(2-phenoxyethyl)-1H-1,2,4-triazole-1-ethanol,
α-(2,4-dichlorophenyl)-β-fluoro-b-propyl-1H-1,2,4-triazole-1-ethanol,
α-(2,4-dichlorophenyl)-β-methoxy-a-methyl-1H-1,2,4-triazole-1-ethanol,
α-(5-methyl-1,3-dioxan-5-yl)-β-[[4-(trifluoromethyl)-phenyl]-methylene]-1H-1,2,4-triazole-1-ethanol,
(5RS,6RS)-6-hydroxy-2,2,7,7-tetramethyl-5-(1H-1,2,4-triazol-1-yl)-3-octanone,
(E)-a-(methoxyimino)-N-methyl-2-phenoxy-phenylacetamide,
1-isopropyl{2-methyl-1-[[[1-(4-methylphenyl)-ethyl]-amino]-carbonyl]-propyl}-carbamate,
1-(2,4-dichlorophenyl)-2-(1H-1,2,4-triazol-1-yl)-ethanone-O-(phenylmethyl)-oxime,
1-(2-methyl-1-naphthalenyl)-1H-pyrrole-2,5-dione,
1-(3,5-dichlorophenyl)-3-(2-propenyl)-2,5-pyrrolidinedione,
1-[(diiodomethyl)-sulphonyl]-4-methyl-benzene,
1-[[2-(2,4-dichlorophenyl)-1,3-dioxolan-2-yl]-methyl]-1H-imidazole,
1-[[2-(4-chlorophenyl)-3-phenyloxiranyl]-methyl]-1H-1,2,4-triazole,
1-[1-[2-[(2,4-dichlorophenyl)-methoxy]-phenyl]-ethenyl]-1H-imidazole,
1-methyl-5-nonyl-2-(phenylnethyl)-3-pyrrolidinole,
2′,6′-dibromo-2-methyl-4′-trifluoromethoxy-4′-trifluoromethyl-1,3-thiazole-5-carboxanilide,
2,2-dichloro-N-[1-(4-chlorophenyl)-ethyl]-1-ethyl-3-methyl-cyclopropanecarboxamide,
2,6-dichloro-5-(methylthio)-4-pyrimidinyl-thiocyanate,
2,6-dichloro-N-(4-trifluoromethylbenzyl)-benzamide,
2,6-dichloro-N-[[4-(trifluoromethyl)-phenyl]-methyl]-benzamide,
2-(2,3,3-triiodo-2-propenyl)-2H-tetrazole,
2-[(1-methylethyl)-sulphonyl]-5-(trichloromethyl)-1,3,4-thiadiazole,
2-[[6-deoxy-4-O-(4-O-methyl-β-D-glycopyranosyl)-a-D-glucopyranosyl]-amino]-4-methoxy-1H-pyrrolo[2,3-d]pyrimidine-5-carbonitrile,
2-aminobutane,
2-bromo-2-(bromomethyl)-pentanedinitrile,
2-chloro-N-(2,3-dihydro-1,1,3-trimethyl-1H-inden-4-yl)-3-pyridinecarboxamide,
2-chloro-N-(2,6-dimethylphenyl)-N-(isothiocyanatomethyl)-acetamide,
2-phenylphenol (OPP),
3,4-dichloro-1-[4-(difluoromethoxy)-phenyl]-1H-pyrrole-2,5-dione,
3,5-dichloro-N-[cyano[(1-methyl-2-propynyl)-oxy]-methyl]-benzamide,
3-(1,1-dimethylpropyl)-1-oxo-1H-indene-2-carbonitrile,
3-[2-(4-chlorophenyl)-5-ethoxy-3-isoxazolidinyl]-pyridine,
4-chloro-2-cyano-N,N-dimethyl-5-(4-methylphenyl)-1H-imudazole-1-sulphonamide,
4-methyl-tetrazolo[1,5-a]quinazolin-5(4H)-one,
8-(1,1-dimethylethyl)-N-ethyl-N-propyl-1,4-dioxaspiro[4.5]decane-2-methanamine,
8-hydroxyquinoline sulphate,
9H-xanthene-2-[(phenylamino)-carbonyl]-9-carboxylic hydrazide,
bis-(1-methylethyl)-3-methyl-4-[(3-methylbenzoyl)-oxy]-2,5-thiophenedicarboxylate,
cis-1-(4-chlorophenyl)-2-(1H-1,2,4-triazol-1-yl)-cycloheptanol,
cis-4-[3-[4-(1,1-dimethylpropyl)-phenyl-2-methylpropyl]-2,6-dimethyl-morpholine hydrochloride,
ethyl[(4-chlorophenyl)-azo]-cyanoacetate,
potassium bicarbonate,
methanetetrathiol-sodium salt,
methyl 1-(2,3-dihydro-2,2-dimethyl-1H-inden-1-yl)-1H-imidazole-5-carboxylate,
methyl N-(2,6-dimethylphenyl)-N-(5-isoxazolylcarbonyl)-DL-alaninate,
methyl N-(chloroacetyl)-N-(2,6-dimethylphenyl)-DL-alaninate,
N-(2,3-dichloro-4-hydroxyphenyl)-1-methyl-cyclohexanecarboxamide,
N-(2,6-dimethylphenyl)-2-methoxy-N-(tetrahydro-2-oxo-3-furanyl)-acetamide,
N-(2,6-dimethylphenyl)-2-methoxy-N-(tetrahydro-2-oxo-3-thienyl)-acetamide,
N-(2-chloro-4-nitrophenyl)-4-methyl-3-nitro-benzenesulphonamide,
N-(4-cyclohexylphenyl)-1,4,5,6-tetrahydro-2-pyrimidineamine,
N-(4-hexylphenyl)-1,4,5,6-tetrahydro-2-pyrimidineamine,
N-(5-chloro-2-methylphenyl)-2-methoxy-N-(2-oxo-3-oxazolidinyl)-acetamide,
N-(6-methoxy)-3-pyridinyl)-cyclopropanecarboxamide,
N-[2,2,2-trichloro-1-[(chloroacetyl)-amino]-ethyl]-benzamide,
N-[3-chloro-4,5-bis(2-propinyloxy)-phenyl]-N′-methoxy-methaneimidamide,
N-formyl-N-hydroxy-DL-alanine-sodium salt,
O,O-diethyl[2-(dipropylamino)-2-oxoethyl]-ethylphosphoramidothioate,
O-methyl S-phenyl phenylpropylphosphoramidothioate,
S-methyl 1,2,3-benzothiadiazole-7-carbothioate,
spiro[2H]-1-benzopyran-2,1′(3′H)-isobenzofuran]-3′-one,
4-[3,4-dimethoxyphenyl)-3-(4-fluorophenyl)-acryloyl]-morpholine.

Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:
abamectin, acephate, acetamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin,
Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,
cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothinidine, cyanophos, cycloprene, cycloprothrin, cyfluthrin, cyhalothrin, cyhexatin, cypermethrin, cyromazine,
deltamethrin, demeton M, demeton S, demeton-S-methyl, diafenthiuron, diazinon, dichlorvos, dicofol, diflubenzuron, dimethoate, dimethylvinphos, diofenolan, disulfoton, docusat-sodium, dofenapyn,
eflusilanate, emamectin, empenthrin, endosulfan, Entomopfthora spp., esfenvalerate, ethiofencarb, ethion, ethoprophos, etofenprox, etoxazole, etrimfos,
fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazinam, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,
granulosis viruses,
halofenozide, HCH, heptenophos, hexaflumuron, hexythiazox, hydroprene,
imidacloprid, indoxacarb, isazofos, isofenphos, isoxathion, ivermectin,
nuclear polyhedrosis viruses,
lambda-cyhalothrin, lufenuron,
malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,
naled, nitenpyram, nithiazine, novaluron,
omethoate, oxamyl, oxydemethon M,
Paecilomyces fumosoroseus, parathion A, parathion M, permethrin, phenthoate; phorate, phosalone, phosmet, phosphamidon, phoxim, pirimicarb, pirimiphos A, pirimiphos M, profenofos, promecarb, propargite, propoxur, prothiofos, prothoate, pymetrozine, pyraclofos, pyresmethrin, pyrethrum, pyridaben, pyridathion, pyrimidifen, pyriproxyfen,
quinalphos,
ribavirin,
salithion, sebufos, silafluofen, spinosad, sulfotep, sulprofos,
tau-fluvalinate, tebufenozide, tebufenpyrad, tebupirimiphos, teflubenzuron, tefluthrin, temephos, temivinphos, terbufos, tetrachlorvinphos, tetradifon, theta-cypermethrin, thiacloprid, thiamethoxam, thiapronil, thiatriphos, thiocyclam hydrogen oxalate, thiodicarb, thiofanox, thuringiensin; tralocythrin, tralomethrin, triarathene, triazamate, triazophos, triazuron, trichlophenidine, trichlorfon, triflumuron, trimethacarb,
vamidothion, vaniliprole, Verticillium lecanii,
YI 5302,
zeta-cypermethrin, zolaprofos,
(1R-cis)-[5-(phenylmethyl)-3-furanyl]-methyl-3-[(dihydro-2-oxo-3(2H)-furanylidene)-methyl]-2,2-dimethylcyclopropanecarboxylate,
(3-phenoxyphenyl)-methyl-2,2,3,3-tetramethylcyclopropanecarboxylate,
1-[(2-chloro-5 -thiazolyl)methyl]tetrahydro-3,5-dimethyl-N-nitro-1,3,5 -triazine-2(1H)-imine,
2-(2-chloro-6-fluorophenyl)-4-[4-(1,1-dimethylethyl)phenyl]-4,5-dihydro-oxazole,
2-(acetyloxy)-3-dodecyl-1,4-naphthalenedione,
2-chloro-N-[[[4-(1-phenylethoxy)-phenyl]-amino]-carbonyl]-benzamide,
2-chloro-N-[[[4-(2,2-dichloro-1,1-difluoroethoxy)-phenyl]-amino]-carbonyl]-benzamide,
3-methylphenyl propylcarbamate,
4-[4-(4-ethoxyphenyl)-4-methylpentyl]-1-fluoro-2-phenoxy-benzene,
4-chloro-2-(1,1-dimethylethyl)-5-[[2-(2,6-dimethyl-4-phenoxyphenoxy)ethyl]thio]-3(2H)-pyridazinone,
4-chloro-2-(2-chloro-2-methylpropyl)-5-[(6-iodo-3-pyridinyl)methoxy]-3(2H)-pyridazinone,
4-chloro-5-[(6-chloro-3-pyridinyl)methoxy]-2-(3,4-dichlorophenyl)-3(2H)-pyridazinone,
Bacillus thuringiensis strain EG-2348,
[2-benzoyl-1-(1,1-dimethylethyl)-hydrazinobenzoic acid,
2,2-dimethyl-3-(2,4-dichlorophenyl)-2-oxo-1-oxaspiro[4.5]dec-3-en-4-yl butanoate,
[3-[(6-chloro-3-pyridinyl)methyl]-2-thiazolidinylidene]-cyanamide,
dihydro-2-(nitromethylene)-2H-1,3-thiazine-3(4H)-carboxaldehyde,
ethyl [2-[[1,6-dihydro-6-oxo-1-(phenylmethyl)-4-pyridazinyl]oxy]ethyl]-carbamate,
N-(3,4,4-trifluoro-1-oxo-3-butenyl)-glycine,
N-(4-chlorophenyl)-3-[4-(difluoromethoxy)phenyl]-4,5-dihydro-4-phenyl-1H-pyrazole-1-carboxamide,
N-[(2-chloro-5-thiazolyl)methyl]-N′-methyl-N″-nitro-guanidine,
N-methyl-N′-(1-methyl-2-propenyl)-1,2-hydrazinedicarbothioamide,
N-methyl-N′-2-propenyl-1,2-hydrazinedicarbothioamide,
O,O-diethyl[2-(dipropylamino)-2-oxoethyl]-ethylphosphoramidothioate,
N-cyanomethyl-4-trifluoromethyl-nicotinamide,
3,5-dichloro-1-(3,3-dichloro-2-propenyloxy)-4-[3-(5-trifluoromethylpyridin-2-yloxy)propoxy]-benzene.

It is also possible to admix other known active compounds, such as herbicides, fertilizers and growth regulators.

When used as insecticides, the active compounds according to the invention can furthermore be present in their commercially available formulations and in the use forms, prepared from these, formulations, as a mixture with synergistic agents. Synergistic agents are compounds which increase the action of the active compounds according to the invention, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having specific properties (“traits”) which have been obtained by conventional breeding, by mutagenesis or by recombinant DNA techniques. This can be varieties, bio- and genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures; increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which extend beyond the effects which were actually to be expected.

The preferred transgenic plants or plant cultivars (i.e. those obtained by genetic engineering) which are to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapevines), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combinations with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these or still to be developed genetic traits, which plants will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or the mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattella germanica and Supella spp.

From the subclass of the Acaria (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) according to the invention can be used as formulations (for example powders, emulsions, free-flowing compositions), which comprise the active compounds according to the invention in an amount of from 1 to 80% by weight, directly or after 100 to 10 000-fold dilution, or they can be used as a chemical bath.

It has furthermore been found that the compounds according to the invention have a strong insecticidal action against insects which destroy industrial materials.

The following insects may be mentioned as examples and as preferred—but without a limitation:

Beetles, such as
Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec. and Dinoderus minutus.

Hymenopterons, such as
Sirex juvencus, Urocerus gigas, Urocerus gigas taignus and Urocerus augur.

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus.

Bristletails, such as Lepisma saccharina.

Industrial materials in the present connection are to be understood as meaning non-living materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agent according to the invention or mixtures comprising it are to be understood as meaning, for example:
building timber, wooden beams, railway sleepers, bridge components, boat jetties, wooden vehicles, boxes, pallets, containers, telegraph poles, wood panelling, wooden window frames and doors, plywood, chipboard, joinery or wooden products which are used quite generally in house-building or in building joinery.

The active compounds according to the invention can be used as such, in the form of concentrates or in generally customary formulations, such as powders, granules, solutions, suspensions, emulsions or pastes.

The formulations mentioned can be prepared in a manner known per se, for example by mixing the active compounds according to the invention with at least one solvent or diluent, emulsifier, dispersing agent and/or binder or fixing agent, a water repellent, if appropriate siccatives and UV stabilizers and if appropriate dyestuffs and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpoint above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., terpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, provided that the solvent mixture likewise has an evaporation number above 35 and a flashpoint above 30° C., preferably above 45° C., and that the insecticide/fungicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-coumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or a drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl)adipate, stearates, such as butyl stearate or amyl stearate, oleates, such as butyl oleate, glycerol ethers or higher molecular weight glycol ethers, glycerol esters and p-toluenesulphonic acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Very particularly preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyfenozide and triflumuron,
and also fungicides, such as epoxyconazole, hexaconazole, azaconazole, propiconazole, tebuconazole, cyproconazole, metconazole, imazalil, dichlorfluanid, tolylfluanid, 3-iodo-2-propinyl-butyl carbamate, N-octyl-isothiazolin-3-one and 4,5-dichloro-N-octylisothiazolin-3-one.

The compounds according to the invention can at the same time be employed for protecting objects which come into contact with salt water or brackish water, in particular hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulphide, antimony oxide, polymeric butyl titanate, phenyl-(bispyridine)-bismuth chloride, tri-n-butyltin fluoride, manganese ethylenebisthiocarbamate, zinc dimethyldithilocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combinations with the antifouling compositions according to the invention are:
algicides such as
2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;
fungicides such as
benzo[b]thiophenecarboxylic acid cyclohexylamide S,S-dioxide, dichlofluanid, fluorfolpet, 3-iodo-2-propynyl butylcarbamate, tolylfluanid and azoles such as azaconazole, cyproconazole, epoxyconazole, hexaconazole, metconazole, propiconazole and tebuconazole;
molluscicides such as
fentin acetate, metaldehyde, methiocarb, niclosamid, thiodicarb and trimethacarb; or conventional antifouling active compounds such as 4,5-dichloro-2-octyl-4-isothiazolin-3-one, diiiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphoriyl)-pyridine, 2,4,5,6-tetrachloroisophthalonitrile, tetramethylthiuram disulphide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds according to the invention are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all development stages. These pests include:

From the order of the Scorpionidea, for example, Buthus occitanus.

From the order of the Acarina, for example, Argas persicus, Argas reflexus, Bryobia spp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus and Dermatophagoides forinae.

From the order of the Araneae, for example, Aviculariidae and Araneidae.

From the order of the Opiliones, for example, Pseudoscorpiones chelifer, Pseudoscorpiones cheiridium and Opiliones phalangium.

From the order of the Isopoda; for example, Oniscus asellus and Porcellio scaber.

From the order of the Diplopoda, for example, Blaniulus guttulatus and Polydesmus spp.

From the order of the Chilopoda, for example, Geophilus spp.

From the order of the Zygentoma, for example, Ctenolepisma spp., Lepisma saccharina and Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa and Supella longipalpa.

From the order of the Saltatoria, for example, Acheta domesticus.

From the order of the Dermaptera, for example, Forficula auricularia.

From the order of the Isoptera, for example, Kalotermes spp. and Reticulitermes spp.

From the order of the Psocoptera, for example, Lepinatus spp. and Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais and Stegobium paniceum. From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans and Tipula paludosa.

From the order of the Lepidoptera, for example, Achroia grisella, Galleria mellonella, Plodia interpunctella, Tinea cloacella, Tinea pellionella and Tineola bisselliella.

From the order of the Siphonaptera, for example, Ctenocephalides canis, Ctenocephalides felis, Pulex irritans, Tunga penetrans and Xenopsylla cheopis.

From the order of the Hymenoptera, for example, Camponotus herculeanus, Lasius fuliginosus, Lasius niger, Lasius umbratus, Monomorium pharaonis, Paravespula spp. and Tetramorium caespitum.

From the order of the Anoplura, for example, Pediculus humanus capitis, Pediculus humanus corporis and Phthirus pubis.

From the order of the Heteroptera, for example, Cimex hemipterus, Cimex lectularius, Rhodinus prolixus and Triatoma infestans.

In the field of household insecticides, they are used alone or in combination with other suitable active compounds, such as phosphates, carbamates, pyrethroids, growth regulators or active compounds from other known classes of insecticide.

They are employed as aerosols, unpressurized spray products, for example pump and atomizer sprays, automatic misting systems, foggers, foams, gels, evaporator products with evaporator tablets made of cellulose or polymer, liquid evaporators, gel and membrane evaporators, propeller-driven evaporators, unpowered, or passive evaporation systems, moth papers, moth bags and moth gels, as granules or dusts, in baits for spreading or in bait stations.

### PREPARATION EXAMPLES

### Example 1

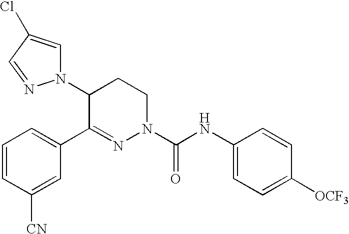

A solution of 1.27 g (4.4 mmol) of 3-(3-cyanophenyl)-4-(4-chloropyrazol-1-yl)-1,4,5,6-tetrahydropyridazine (Ex. II-1) in 30 ml of acetonitrile is admixed dropwise at room temperature with a solution of 0.89 g (4.4 mmol) of 4-trifluoromethoxyphenyl isocyanate in 10 ml of acetonitrile and the mixture is subsequently stirred at room temperature for 6 hours. The precipitated product is then filtered off and the filtrate is concentrated by evaporation in vacuo. The residue is triturated with ethanol and following crystallization the product is filtered off with suction and washed with ethanol.

This gives 0.44 g (20% of theory) of 3-(3-cyanophenyl)-4-(4-chloropyrazol-1-yl)-1-(4-trifluoromethoxyphenylamino)carbonyl-1,4,5,6-tetrahydropyridazine in the form of colourless crystals whose logP (pH 2)=4.08 and whose melting point is 216° C.

Preparation of the Precursors

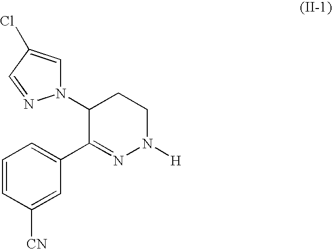

A solution of 5.3 g (17.2 mmol) of 3′-cyano-2-(4-chloropyrazol-1-yl)-4-chlorobutyrophenone (Ex. IV-1) in 50 ml of ethanol is admixed with 1.7 g (35 mmol) of hydrazine hydrate and the mixture is stirred at room temperature overnight. The solvent is then distilled off in vacuo, water is added to the residue, and the mixture is extracted with ethyl acetate. The organic phase is separated off, dried over sodium sulphate and concentrated by evaporation in vacuo.

This gives 3.8 g (78% of theory) of 3-(3-cyanophenyl)-4-(4-chloropyrazol-1-yl)-1,4,5,6-tetrahydropyridazine as a colourless powder whose logP (pH 2)=2.29 and whose melting point is 171° C.

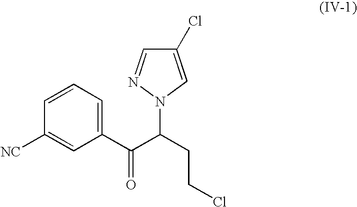

A mixture of 7 g (0.05 mol) of 4-chloropyrazole hydrochloride, 13.8 g (0.1 mol) of potassium carbonate and 60 ml of acetonitrile is admixed dropwise at −5° C. with a solution of 14.3 g (0.05 mol) of 3′-cyano-2-bromo-4-chlorobutyrophenone (Ex. V-1) in 20 ml of acetonitrile and the mixture is subsequently stirred at room temperature for 18 hours. Water is then added to the reaction mixture, which is extracted with ethyl acetate. The organic phase is separated off, dried over sodium sulphate and concentrated by evaporation in vacuo. The residue is triturated with isopropanol and left to stand at room temperature overnight, after which the precipitated product is filtered off with suction.

This gives 3.8 g (25% of theory) of 3′-cyano-2-(4-chloropyrazol-1-yl)-4-chlorobutyrophenone as a beige powder whose logP (pH 2)=2.95.

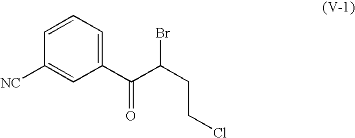

A solution of 32.7 g (0.15 mol) of 3′-cyano-4-chlorobutyrophenone (Ex. VI-1) in 150 ml of methylene chloride is admixed first with 0.1 g of aluminium chloride and then dropwise with 25.6 g (0.16 mol) of bromine. The mixture is subsequently stirred at room temperature overnight and then nitrogen is passed through the reaction mixture in order to remove the hydrogen bromide. Then water is added with stirring, followed by saturated sodium bicarbonate solution, until the aqueous phase is neutral. The organic phase is then separated off, dried over sodium sulphate, filtered and concentrated by evaporation in vacuo. The residue is triturated with petroleum ether and following crystallization the product is filtered off with suction.

This gives 40.8 g (95% of theory) of 3′-cyano-2-bromo-4-chlorobutyrophenone as a beige powder whose logP (pH 2)=3.09.

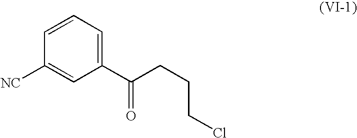

A mixture of 48 g (0.22 mol) of 3-(3-cyanobenzoyl)-γ-butyrolactone (Ex. VIIa-1) and 200 ml of concentrated hydrochloric acid is warmed at 35 to 40° C. for 2 hours with stirring, after which the temperature is raised to 50° C. for 30 minutes. The reaction mixture is cooled in an ice bath and then the precipitated product is filtered off with suction, washed with water and dried in air.

This gives 32.9 g (71% of theory) of 3′-cyano-4-chlorobutyrophenone in the form of a beige powder whose logP (pH 2)=2.49.

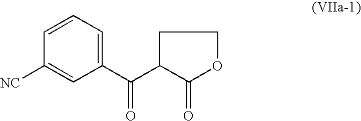

A solution of 30.2 g (0.27 mol) of potassium tert-butoxide in 300 ml of tetrahydrofuran is admixed dropwise at 25-30° C. with a solution of 40.3 g (0.25 mol) of methyl 3-cyanobenzoate and 21.8 g (0.25 mol) of γ-butyrolactone in 50 ml of tetrahydrofuran and the mixture is subsequently stirred at room temperature for 18 hours. The solvent is then distilled off in vacuo, the residue is dissolved in water and the solution is extracted with diethyl ether. The aqueous phase is separated off and adjusted to a pH of about 1-2 using dilute hydrochloric acid, with ice cooling. It is extracted twice with methylene chloride, the organic phases are dried over sodium sulphate and then the solvent is evaporated off in vacuo.

This gives 46.1 g (86% of theory) of 3-(3-cyanobenzoyl)-γ-butyrolactone in the form of a partially crystalline oil whose logP (pH 2)=1.48.

The compounds of the formula (I) indicated in Table 1 below are obtained in analogy to Ex. 1 and/or in accordance with the general preparation instructions:

TABLE 1

| | |
|---|---|
| | (I) |
| 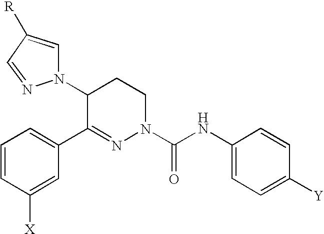 | |

| | | | | logP (pH 2)/ |
|---|---|---|---|---|
| Ex. No. | R | X | Y | m.p. [° C.] |
| 2 | Cl | CF₃ | Cl | 4.60/199 |
| 3 | Cl | CF₃ | OCF₃ | 4.83/192 |
| 4 | Cl | F | CF₃ | 4.37/194 |
| 5 | Cl | F | Cl | 4.18/213 |
| 6 | Cl | F | OCF₃ | 4.43/148 |
| 7 | Cl | Cl | OCF₃ | 4.73/181 |
| 8 | Cl | Cl | Cl | 4.49/203 |
| 9 | Cl | Cl | CF₃ | 4.69/199 |
| 10 | Cl | OCF₃ | Cl | 4.73/158 |
| 11 | Cl | Br | Cl | 4.58/198 |
| 12 | Cl | Br | OCF₃ | 4.82/197 |
| 13 | Cl | Br | CF₃ | 4.77/196 |
| 14 | Cl | CF₃ | CF₃ | 4.79/176 |
| 15 | Cl | F | SCF₃ | 4.81/166 |
| 16 | Cl | CN | CF₃ | 4.03/229 |
| 17 | Cl | CN | SCF₃ | 4.44/182 |
| 18 | CN | Cl | Cl | 3.90/228 |
| 19 | CN | Cl | OCF₃ | 4.15/186 |
| 20 | Cl | Cl | CN | 3.77 |
| 21 | CN | Cl | SCF₃ | 4.53/194 |
| 22 | CN | Br | Cl | 3.96/213 |
| 23 | Cl | CF₃ | SCF₃ | 5.21/188 |
| 24 | CN | Br | SCF₃ | 4.64/206 |
| 25 | CN | Br | OCF₃ | 4.24/192 |
| 26 | CN | CN | SCF₃ | 3.94/242 |
| 27 | CN | Br | CF₃ | 4.18/244 |
| 28 | CN | Cl | Br | 4.03 |
| 29 | CN | CN | OCF₃ | 3.62/239 |
| 30 | OCH₃ | Cl | OCF₃ | |
| 31 | OCF₃ | Cl | OCF₃ | |
| 32 | SCH₃ | Cl | OCF₃ | |
| 33 | COOCH₃ | Cl | CF₃ | |
| 34 | COOCH₃ | Cl | OCF₃ | |
| 35 | COOCH₃ | Cl | SCF₃ | |
| 36 | COOC₂H₅ | Cl | OCF₃ | |
| 37 | COOCH₃ | Br | OCF₃ | |
| 38 | CONH₂ | Cl | OCF₃ | |
| 39 | CON(CH₃)₂ | Cl | OCF₃ | |
| 40 | CONHCH₃ | Cl | OCF₃ | |
| 41 | CN | SCH₃ | OCF₃ | |
| 42 | CN | SO₂CH₃ | OCF₃ | |
| 43 | CN | SO₂CF₃ | OCF₃ | |
| 44 | Cl | SO₂CF₃ | OCF₃ | |
| 45 | CN | SO₂CF₃ | Cl | |
| 46 | CN | SO₂CF₃ | SCF₃ | |
| 47 | CN | SO₂CF₃ | CF₃ | |
| 48 | Br | CN | OCF₃ | |
| 49 | OCF₃ | Cl | OCF₃ | |
| 50 | CN | SCF₃ | OCF₃ | |
| 51 | Cl | Cl | SO₂CF₃ | |
| 52 | CN | Cl | SO₂CF₃ | |
| 53 | CN | Br | SO₂CF₃ | |
| 54 | Cl | CN | SO₂CF₃ | |
| 55 | CN | CF₃ | SO₂CF₃ | |
| 56 | CN | OCF₃ | SO₂CF₃ | |
| 57 | Br | Cl | SO₂CF₃ | |
| 58 | CN | CF₃ | OCF₃ | |
| 59 | CN | OCF₃ | OCF₃ | |
| 60 | CN | CF₃ | SCF₃ | |
| 61 | CN | OCF₃ | SCF₃ | |

The novel precursors of the formula (II) indicated in Table 2 below may be obtained in analogy to Example 1:

TABLE 2

| | |
|---|---|
| | (II) |
| 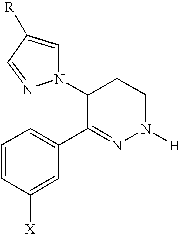 | |

| Ex. No. | R | X | logP (pH 2) |
|---|---|---|---|
| II-2 | Cl | CF₃ | 3.09 |
| II-3 | Cl | F | 2.59 |
| II-4 | Cl | Cl | 2.93 |
| II-5 | Cl | OCF₃ | 3.32 |
| II-6 | Cl | Br | 3.03 |
| II-7 | CN | Cl | 2.40 |
| II-8 | CN | Br | 2.49 |
| II-9 | CN | CN | 1.83 |
| II-10 | OCH₃ | Cl | |
| II-11 | OCF₃ | Cl | |
| II-12 | SCF₃ | Cl | |
| II-13 | COOCH₃ | Cl | |
| II-14 | COOCH₃ | Br | |
| II-15 | CONH₂ | Cl | |
| II-16 | CON(CH₃)₂ | Cl | |
| II-17 | CONHCH₃ | Cl | |
| II-18 | CN | SCH₃ | |
| II-19 | CN | SO₂CH₃ | |
| II-20 | CN | SO₂CF₃ | |
| II-21 | Cl | SO₂CF₃ | |
| II-22 | Br | CN | |
| II-23 | OCF₃ | Cl | |
| II-24 | Cl | SCF₃ | |
| II-25 | CN | SCF₃ | |
| II-26 | Cl | SCH₃ | |
| II-27 | CN | CF₃ | |
| II-28 | CN | OCF₃ | |

The novel intermediates of the formula (IV) indicated in Table 3 below may be obtained in analogy to Example 1:

TABLE 3

| | |
|---|---|
| | (IV) |
| 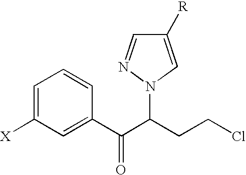 | |

| Ex. No. | R | X | logP (pH 2) |
|---|---|---|---|
| IV-2 | Cl | CF₃ | 3.84 |
| IV-3 | Cl | F | 3.31 |
| IV-4 | Cl | Cl | 3.68 |
| IV-5 | Cl | OCF₃ | 3.99 |
| IV-6 | Cl | Br | 3.78 |
| IV-7 | CN | Cl | 3.06 |
| IV-8 | CN | Br | 3.13 |
| IV-9 | CN | CN | 2.41 |
| IV-10 | OCH₃ | Cl | |
| IV-11 | OCF₃ | Cl | |
| IV-12 | SCF₃ | Cl | |
| IV-13 | COOCH₃ | Cl | |
| IV-14 | COOCH₃ | Br | |
| IV-15 | CONH₂ | Cl | |
| IV-16 | CON(CH₃)₂ | Cl | |
| IV-17 | CONHCH₃ | Cl | |
| IV-18 | CN | SCH₃ | |
| IV-19 | CN | SO₂CH₃ | |
| IV-20 | CN | SO₂CF₃ | |
| IV-21 | Cl | SO₂CF₃ | |
| IV-22 | Br | CN | |
| IV-23 | OCF₃ | Cl | |
| IV-24 | Cl | SCF₃ | |
| IV-25 | CN | SCF₃ | |
| IV-26 | Cl | SCH₃ | |
| IV-27 | CN | CF₃ | |
| IV-28 | CN | OCF₃ | |

The intermediates of the formula (V) indicated in Table 4 below may be obtained in analogy to Example 1:

TABLE 4

| | |
|---|---|
| | (V) |
| 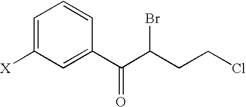 | |

| Ex. No. | X | logP (pH 2) |
|---|---|---|
| V-2 | F | 3.47 |
| V-3 | OCF₃ | 4.19 |
| V-4 | Br | 4.02 |
| V-5 | SCF₃ | |
| V-6 | SO₂CF₃ | |
| V-7 | SCH₃ | |

The intermediates of the formula (VI) indicated in Table 5 below may be obtained in analogy to Example 1:

TABLE 5

| | |
|---|---|
| | (VI) |
| 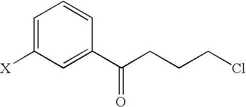 | |

| Ex. No. | X | logP (pH 2) |
|---|---|---|
| VI-2 | OCF₃ | 3.71 |
| VI-3 | Br | 3.50 |
| VI-4 | SCF₃ | |
| VI-5 | SO₂CF₃ | |
| VI-6 | SCH₃ | |

The novel intermediates of the formula (VIIa) indicated in Table 6 below may be obtained in analogy to Example 1:

TABLE 6

| | |
|---|---|
| | (VIIa) |
| 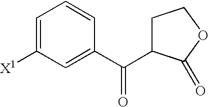 | |

| Ex. No. | X¹ | logP (pH 2) |
|---|---|---|
| VIIa-2 | CF₃ | 2.42 |
| VIIa-3 | F | 1.77 |
| VIIa-4 | OCF₃ | 2.59 |
| Vlla-5 | Br | 2.22 |
| VIIa-6 | SCF₃ | |
| VIIa-7 | SO₂CF₃ | |
| VIIa-8 | SCH₃ | |

### USE EXAMPLES

### Example A

Heliothis virescens Test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Soya shoots (Glycine max) are treated by being dipped into the preparation of active compound at the desired concentration and are populated with Heliothis virescens caterpillars while the leaves are still moist.

After the desired period of time, the kill in % is determined. 100% means that all of the caterpillars have been killed: 0% denotes that no caterpillars have been killed.

In this test, at an exemplary active compound concentration of 500 ppm, for example, the compounds from Preparation Examples 1, 13, 14, 15 and 17 show a kill of 100% after 6 days.

### Example B

Phaedon larvae Test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound at the desired concentration and are populated with larvae of the mustard beetle (Phaedon cochleariae) while the leaves are still moist.

After the desired period of time, the kill in % is determined. 100% means that all of the beetle larvae have been killed: 0% denotes that no beetle larvae have been killed.

In this test, at an exemplary active compound concentration of 1000 ppm, for example, the compounds from Preparation Examples 2, 3, 4, 5, 6, 7, 8, 9, 10, 11, 12 and 16 show a kill of 100% after 7 days.

### Example C

Plutella test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound at the desired concentration and are populated with caterpillars of the cabbage moth (Plutella xylostella) while the leaves are still moist.

After the desired period of time, the kill in % is determined. 100% means that all of the caterpillars have been killed: 0% denotes that no caterpillars have been killed.

In this test, at an exemplary active compound concentration of 500 ppm, for example, the compounds from Preparation Examples 1, 13, 14, 15 and 17 show a kill of 100% after 6 days.

### Example D

Spodoptera exigua Test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound at the desired concentration and are populated with caterpillars of the army worm (Spodoptera exigua) while the leaves are still moist.

After the desired period of time, the kill in % is determined. 100% means that all of the caterpillars have been killed: 0% denotes that no caterpillars have been killed.

In this test, at an exemplary active compound concentration of 500 ppm, for example, the compounds from Preparation Examples 1, 13, 14, 15 and 17 show a kill of 100% after 6 days.

### Example E

Spodoptera frugiperda Test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound at the desired concentration and are populated with caterpillars of the army worm (Spodoptera frugiperda) while the leaves are still moist.

After the desired period of time, the kill in % is determined. 100% means that all of the caterpillars have been killed: 0% denotes that no caterpillars have been killed.

In this test, at an exemplary active compound concentration of 1000 ppm, for example, the compounds from Preparation Examples 2, 3, 4, 5, 6, 7, 8, 9, 10, 11, 12 and 16 show a kill of 100% after 7 days.

### Example F

Activity Duration Test: Heliothis virescens

| | |
|---|---|
| Solvent: | 9 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cotton plants (Gossypium hirsutum) are sprayed with a preparation of the active compound in the desired concentration. After the number of days indicated, Heliothis virescens caterpillars are placed onto the treated leaves in infection chambers.

After the desired period of time, the kill in % is determined. 100% means that all of the caterpillars have been killed: 0% denotes that no caterpillars have been killed.

In this test, at an exemplary active compound concentration of 20 ppm, for example, the compound from Preparation Example 14 shows in each case a kill of 100% on infection after 5, 12, 19 and 26 days.

### Example G

Activity Duration Test: Spodoptera frugiperda

| | |
|---|---|
| Solvent: | 9 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cotton plants (Gossypium hirsutum) are sprayed with a preparation of the active compound in the desired concentration. After the number of days indicated, caterpillars of the army worm (Spodoptera frugiperda) are placed onto the treated leaves in infection chambers.

After the desired period of time, the kill in % is determined. 100% means that all of the caterpillars have been killed: 0% denotes that no caterpillars have been killed.

In this test, at an exemplary active compound concentration of 20 ppm, for example, a good activity is shown as follows:

Degree of kill in %

| Infection after | Ex. No. 14 | Ex. No. 15 |
|---|---|---|
| 5 days | 90 | 90 |
| 12 days | 80 | 90 |
| 19 days | 70 | 80 |
| 26 days | 90 | 90 |

### Example H

Test with Flies (Musca domestica)

| | |
|---|---|
| Test | adult Musca domestica, strain: Reichswald (OP, |
| organisms: | SP, carbamate resistant) |
| Solvent: | Dimethyl sulphoxide |

20 mg of active substance are dissolved in 1 ml of dimethyl sulphoxide; lower concentrations are prepared by dilution with distilled water.

2 ml of this active compound preparation are pipetted onto filter papered disks (Ø 9.5 cm) located in Petri dishes of corresponding size. After the filter disks have dried, 25 test organisms are transferred to the Petri dishes and covered.

The activity of the active compound preparation is determined after 1, 3, 5, 24 and 48 hours. 100% means that all the flies were killed; 0% means that none of the flies was killed.

In this test a good effect is exhibited, for example, by the following compounds from the Preparation Examples:

| Ex. | Concentration μg | % |
|---|---|---|
| No. | fly | Activity/Kill |
| 9 | 100/20/4 | 95/85/0 |
| 7 | 100/20/4 | 99/50/0 |
| 2 | 100/20/4 | 95/30/0 |
| 3 | 100/20/4 | 95/50/0 |
| 6 | 100/20/4 | 99/50/0 |
| 23 | 100/20/4 | 90/0 |

### Example I

Blowfly Larvae Test/Development Inhibition Activity

| | |
|---|---|
| Test organisms: | Lucilia cuprina larvae |
| Solvent: | Dimethyl sulphoxide |

20 mg of active substance are dissolved in 1 ml of dimethyl sulphoxide; lower concentrations are prepared by dilution with distilled water.

About 20 Lucilia cuprina larvae are introduced into a test tube containing 1 cm³ of horse meat and 0.5 ml of the test preparation of active compound. The efficacy of the active compound preparation is determined after 24 and 48 hours. The test tubes are transferred to beakers whose bases are covered with sand. After 2 more days, the test tubes are removed and the pupae are counted.

The activity of the active compound preparation is assessed in accordance with the number of flies which have hatched following 1.5 times the development period of an untreated control. 100% means that no flies have hatched; 0% means that all of the flies hatched normally.

In this test a good effect is exhibited, for example, by the following compounds from the Preparation Examples:

| Ex. | | % |
|---|---|---|
| No. | Concentration ppm | Activity/Kill |
| 9 | 100/20 | 100/0 |
| 7 | 100/20/4 | 100/100/0 |
| 2 | 100/20/4 | 100/100/0 |
| 3 | 100/20/4 | 100/100/0 |
| 5 | 100/20 | 100/0 |
| 6 | 100/20 | 100/0 |
| 23 | 100/20 | 100/0 |

1. A method for the quantitative determination of an ion in a fluid which comprises subjecting the fluid to voltammetry using an electrode which comprises an electrically conducting support possessing a coating of a solid capable of selecting ions when electrochemically induced during operation.

2. A method according to claim 1 wherein the solid is redox active material.

3. A method according to claim 1 wherein the solid undergoes a reversible ingress/egress of ions when electrochemically induced.

4. A method according to claim 3 wherein the solid undergoes a selective reversible ingress/egress of ions when electrochemically induced.

5. A method according to claim 4 wherein the solid is a synmetal.

6. A method according to claim 1 wherein the solid is either the donor or acceptor of a charge transfer salt pair.

7. A method according to claim 6 wherein the solid is tetracyanoquinodimethane or tetrathiafulvalene or an analogue or derivative thereof.

8. A method according to claim 1 wherein the support is formed of a metal or carbon.

9. A method according to claim 1 wherein the solid coating has an ion exchange membrane over it, the membrane being capable of exchanging ions of the same charge as those capable of being intercalated by the solid.

10. A method according to claim 1 wherein the fluid contains more than one anion or cation and the solid is one which provides the more or most positive mid point potential for the cation to be determined in a mixture of cations or the more negative mid point potential for the anion to be determined in a mixture of anions.

11. A method according to claim 1 wherein the fluid is placed in a cell comprising the electrode, a counter electrode for supplying a potential difference and a reference electrode.

12. A method according to claim 11 wherein the cell comprises a microelectrode.

13. A method according to claim 1 wherein the electrode is reset following a determination by voltammetric cycling.

14. (canceled)

15. A device for the quantitative determination of an ion in a fluid by subjecting the fluid to voltammetry, the device comprising an electrode having an electrically conducting support that possesses a coating of a solid capable of selecting ions when electrochemically induced during operation of the device.

16. A device for the quantitative determination of an ion in a fluid which device comprises a receptacle for said fluid, the receptacle comprising an electrode that possesses a coating of a solid capable of selecting ions when electrochemically induced during operation of the device, and a counter electrode for supplying a potential difference.

17. A device according to claim 31 wherein the electrode, the counter electrode and the reference electrode comprise three conductive tracks on a single support.

18. A device according to claim 16 which comprises more than one electrode comprising an electrically conducting support possessing a coating of a solid capable of selecting ions when electrochemically induced during operation.

19. A support comprising at least two conductive tracks, one track of which forming an electrode having a coating of a solid capable of selecting ions when electrochemically induced during operation and another track of which forming a counter electrode for supplying a potential difference, and the support further comprising a reference electrode.

20. A support according to claim 19 comprising a reference electrode as a third track.

21. An electrode which comprises an electrically conducting support possessing a coating of a solid capable of intercalation of ions when electrochemically induced during operation, said solid not being tetracyanoquinodimethane or tetrathiafulvalene.

22. (canceled)

23. A method according to claim 15 wherein the solid is a redox active material.

24. A device according to claim 15 wherein the solid undergoes a reversible ingress/egress of ions when electrochemically induced.

25. A device according to claim 15 wherein the solid undergoes a selective reversible ingress/egress of ions when electrochemically induced.

26. A device according to claim 15 wherein the solid is a synmetal.

27. A device according to claim 15 wherein the solid is either the donor or acceptor of a charge transfer salt pair.

28. A device according to claim 27 wherein the solid is tetracyanoquinodimethane or tetrathiafulvalene or an analogue or derivative thereof.

29. A device according to claim 15 wherein the support is formed of a metal or carbon.

30. A device according to claim 15 wherein the coating has an ion exchange membrane over it, the membrane being capable of exchanging ions of the same charge as those capable of being intercalated by the solid.

31. A device according to claim 16 further comprising a reference electrode.

32. A device according to claim 15 wherein the device comprises a microelectrode.
